(19) United States
(12) Patent Application Publication
(10) Pub. No.: US 2001/0031468 A1
(43) Pub. Date: Oct. 18, 2001

(54) Analyte assays employing universal arrays

(76) Inventors: Alex Chenchik, Palo Alto, CA (US); Grigoriy S. Tchaga, Newark, CA (US); Peter N. Simonenko, Mountain View, CA (US)

Correspondence Address: Bret Field, BOZICEVIC, FIELD & FRANCIS LLP, 200 Middlefield Road, Suite 200, Menlo Park, CA, 94025, US

(21) Appl. No.: 09/752,292

(22) Filed: Dec. 28, 2000

Related U.S. Application Data
(60) Provisional application No. 60/181,366, filed on Feb. 8, 2000.

(51) Int. Cl. C12Q 1/68; G01N 33/53; G01N 33/537; G01N 33/543

(52) U.S. Cl. 435/6; 435/7.92

(57) ABSTRACT

Analyte detection assays, as well as kits, primers and universal arrays for use in practicing the same, are provided. In many embodiments of the subject assays, a population of tagged affinity ligands is first contacted with a sample being assayed under conditions sufficient to produce binding complexes of tagged affinity ligand/analyte complexes between affinity ligands and their corresponding target analytes present in the sample. The resultant composition is then contacted with a universal array of tag complements under hybridization conditions and the presence of any resultant hybridized or surface bound tagged affinity ligand/analyte-tag complement structures is detected. The subject methods find use in a number of different applications, and are particularly suited for use in proteomics.

### CROSS REFERENCE TO RELATED APPLICATIONS

Pursuant to 35 U.S.C. § 119 (e), this application claims priority to the filing date of the U.S. Provisional Patent Application Ser. No. 60/181,366 filed Feb. 8, 2000, the disclosure of which is herein incorporated by reference.

### INTRODUCTION

1. Technical Field

The field of this invention is binding agent arrays, particularly protein arrays, e.g. for use in proteomics.

2. Background of the Invention

Binding agent arrays have become an increasingly important tool in the biotechnology industry and related fields. Binding agent arrays, in which a plurality of binding agents are displayed on a solid support surface in the form of an array or pattern, find use in a variety of applications. One important type of binding agent array is a protein array.

Protein arrays find use in a variety of applications, and are particularly suited for use in proteomics applications. Proteomics involves the qualitative and quantitative measurement of gene activity by detecting and quantitating expression at the protein level, rather than at the messenger RNA level. Proteomics also involves the study of non-genome encoded events, including the post-translational modification of proteins, interactions between proteins, and the location of proteins within a cell. The structure, function, or level of activity of the proteins expressed by the cell are also of interest. Essentially, proteomics inolves the study of part or all of the status of the total protein contained within or secreted by a cell. Proteomics is of increasing interest for a number of reasons, including the fact that measuring the mRNA abundances of a cell potentially provides only an indirect and incomplete assessment of the protein content of the cell, as the level of active protein that is produced in a cell is often determined by factors other than the amount of mRNA produced, e.g. post-translational modifications, etc.

While a number of different protein array formats have been developed for use in proteomics and related applications, the formats developed to date are not without problems. Problems experienced with currently available formats include production issues due to potential inactivation of the protein upon attachment to the support surface, storage stability, changes in binding activity of the protein due to attachment to the support surface, performing the binding reaction at a solid/liquid interface, etc.

As such, there is continued interest in the development of new array formats and protocols that preferably overcome one or more of the above disadvantages often experienced with currently available formats.

Relevant Literature

U.S. patents of interest include: U.S. Pat. Nos. 5,143,854; 5,445,934; 5,556,752; 5,700,637; 5,763,175; 5,807,522; 5,863,722; and 5,994,076. Also of interest are: WO 99/31267; WO 00/04382; WO 00/04389; WO 00/04390; WO 97/24455; WO 98/53103 and WO 99/35289. References of interest include: Southern, et al. Nature Genet. (1999) 21:5-9; Lipshutz, et al., Nature Genet. 1999, 21:20-24; Duggan, et al., Nature Genet. (1999) 21:10-14; and Brown, P. O., Nature Genet (1999) 21:33-37.

### SUMMARY OF THE INVENTION

Analyte detection assays, as well as kits, primers and universal arrays for use in practicing the same, are provided. In many embodiments of the subject assays, a population of tagged affinity ligands is first contacted with a sample being assayed under conditions sufficient to produce binding complexes of tagged affinity ligand/analyte complexes between affinity ligands and their corresponding target analytes present in the sample. The resultant composition is then contacted with a universal array of tag complements under hybridization conditions and the presence of any resultant hybridized or surface bound tagged affinity ligand/analyte-tag complement structures is detected. The subject methods find use in a number of different applications, and are particularly suited for use in proteomics.

### DEFINITIONS

The term “nucleic acid” as used herein means a polymer composed of nucleotides, e.g. naturally occurring deoxyribonucleotides or ribonucleotides, as well as synthetic mimetics thereof which are also capable of participating in sequence specific, Watson-Crick type hybridization reactions, such as is found in peptide nucleic acids, etc.

The term “peptide” as used herein refers to any compound produced by amide formation between a carboxyl group of one amino acid and an amino group of another group.

The term “oligopeptide” as used herein refers to peptides with fewer than about 10 to 20 residues, i.e. amino acid monomeric units.

The term “polypeptide” as used herein refers to peptides with more than 10 to 20 residues.

The term “protein” as used herein refers to polypeptides of specific sequence of more than about 50 residues.

The term “tag” refers to a nucleic acid which has a sequence that is the complement of a tag-complement nucleic acid on an array employed in the subject methods.

The term “tag-complement” refers to a nucleic acid that is the complement of a tag nucleic acid.

The term “affinity ligand” refers to any molecule or compound that has a binding affinity for a target analyte, e.g. a target protein, where the binding affinity is at least about 10⁻⁴ M, usually at least about 10⁻⁶ M. Representative affinity ligands include, but are not limited to, antibodies, as well as binding fragments and mimetics thereof.

The term “non-specific hybridization” refers to the non-specific binding or hybridization of a tag nucleic acid to a tag-complement nucleic acid present on the array surface, where the tag and the tag complement are not substantially complementary.

### DESCRIPTION OF THE SPECIFIC EMBODIMENTS

Analyte detection assays, as well as kits, primers and universal arrays for use in practicing the same, are provided. In many embodiments of the subject assays, a population of tagged affinity ligands is first contacted with a sample being assayed under conditions sufficient to produce binding complexes of tagged affinity ligand/analyte complexes between affinity ligands and their corresponding target analytes present in the sample. The resultant composition is then contacted with a universal array of tag complements under hybridization conditions and the presence of any resultant hybridized or surface bound tagged affinity ligand/analyte-tag complement structures is detected. The subject methods find use in a number of different applications, and are particularly suited for use in proteomics. In further describing the subject invention, the subject methods are discussed first, followed by a review of representative applications in which the subject methods find use as well as a discussion of kits for use in practicing the subject methods.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Methods

As summarized above, the subject invention provides methods for performing analyte detection assays, and more particularly array based hybridization analyte screening, particularly protein screening, assays with a “universal array.” By “array based hybridization analyte screening” is meant an assay or test protocol in which a nucleic acid array, i.e. a plurality of distinct probe nucleic acids stably associated or immobilized on the surface of a solid support (e.g. rigid or flexible solid support), is employed and one or more hybridization interactions occur, i.e. one or more specific Watson-Crick or analogous base pairing interactions between complementary nucleic acid molecules, i.e. tag complement nucleic acids immobilized on the array surface and tag nucleic acids of tagged affinity ligands present in solution. For purposes of convenience in describing the invention, the assays are herein described in terms of hybridization interactions between tag complement and tag nucleic acids, where the tag complement nucleic acids are those stably associated with the surface of the solid support and the tag nucleic acids are tag nucleic acids of the tagged affinity ligands, where the tag nucleic acids hybridize to the array surface if their complement nucleic acid is present on the array surface as a tag complement nucleic acid. In other words, the subject invention provides methods of performing nucleic acid array hybridization assays between an array of tag complement nucleic acids stably associated with or immobilized on the surface of a solid support and a solution of tagged affinity ligands.

While the subject methods are suitable for use in screening a composition for the presence of, and determining the amount of, one or more analytes of interest, where a variety of analytes may be detected, e.g. nucleic acids, proteins, polysaccharides, small molecules, etc., the subject methods are particularly suited for use in detecting the presence of, and determining the amounts of, one or more proteins in a sample. As such and for ease of illustration, the subject methods will now be discussed in terms of protein screening assays, i.e. in terms of those embodiments where the analyte(s) of interest is a protein or polypeptide. However, it is readily within the ability of those of skill in the art to modify the below described methods for use in assays of non-protein analytes, e.g. by changing the nature of the affinity ligand to one that specifically binds to a non-protein analyte.

A feature of the subject invention is that, in practicing the subject array based hybridization assays, a population or plurality of distinct tagged affinity ligands is contacted with an array of tag complements. As such, in practicing the subject methods an array of a plurality of distinct tag complements is contacted with a population or plurality of tagged affinity ligands. In addition, each tag and tag complement in a given population of tag-tag complement pairs employed in the subject assays is chosen to provide substantially uniform hybridization efficiency and substantially no cross-hybridization. In further describing this feature of the subject methods, the population of tagged affinity ligands (and its preparation) will be described first, followed by a description of the tag complement arrays (and methods for their preparation). Finally, further detail regarding the hybridization efficiency and the low cross-hybridization characteristics of the tag-tag complements employed in the subject methods will be provided.

Population of Tagged Affinity Ligands and Methods for its Production

As mentioned above, the subject methods employ a population of distinct tagged affinity ligands. By population is meant a plurality, where the number of tagged affinity ligands in a given population is generally at least about 10, usually at least about 20 and often at least about 50, wherein in many embodiments the number of distinct tagged affinity ligands in a given population may be at least about 100, 200 or higher. In general, the number of distinct tagged affinity ligands in a given population does not exceed about 5,000 and usually does not exceed about 2,000. Any two tagged affinity ligands are considered to be distinct if they include at least one of a different affinity ligand or a different nucleic acid tag. Any two nucleic acids tags are considered to be different if they include a stretch or domain of nucleotides of at least about 20 nt, usually at least about 15 nt and more usually at least about 10 nt which are non-homologous, i.e. have a homology as determined by BLAST using default settings of less than about 80%, preferably less than about 60% and more preferably less than about 50%. Any two affinity ligands are considered distinct if they have a different molecular composition and/or bind to different proteins/polypeptides or other analytes.

By tagged affinity ligand is meant a conjugate molecule that includes an affinity ligand conjugated to a tag nucleic acid, where the two components are generally (though not necessarily) covalently joined to each other, e.g. directly or through a linking group. In other words, in many embodiments the tagged affinity ligand is made up of an affinity ligand covalently joined to a tag nucleic acid, either directly or through a linking group, where the linking group may or may not be cleavable, e.g. enzymatically cleavable (for example, it may include a restriction endonuclease recognized site), photo labile, etc.

Affinity Ligand

The affinity ligand domain, moiety or component of the tagged affinity ligands is a molecule that has a high binding affinity for a target protein. By high binding affinity is meant a binding affinity of at least about 10⁻⁴ M, usually at least about 10⁻⁶ M. The affinity ligand may be any of a variety of different types of molecules, so long as it exhibits the requisite binding affinity for the target protein when present as tagged affinity ligand. As such, the affinity ligand may be a small molecule or large molecule ligand. By small molecule ligand is meant a ligand ranging in size from about 50 to 10,000 daltons, usually from about 50 to 5,000 daltons and more usually from about 100 to 1000 daltons. By large molecule is meant a ligand ranging in size from about 10,000 daltons or greater in molecular weight.

The small molecule may be any molecule, as well as binding portion or fragment thereof, that is capable of binding with the requisite affinity to the target protein. Generally, the small molecule is a small organic molecule that is capable of binding to the protein target of interest. The small molecule will include one or more functional groups necessary for structural interaction with the target protein, e.g. groups necessary for hydrophobic, hydrophilic, electrostatic or even covalent interactions, depending on the particular drug and its intended target. Where the target is a protein, the drug moiety will include functional groups necessary for structural interaction with proteins, such as hydrogen bonding, hydrophobic-hydrophobic interactions, electrostatic interactions, etc., and will typically include at least an amine, amide, sulfhydryl, carbonyl, hydroxyl or carboxyl group, preferably at least two of the functional chemical groups. As described in greater detail below, the small molecule will also comprise a region that may be modified and/or participate in covalent linkage to the tag component of the tagged affinity ligand, without substantially adversely affecting the small molecule's ability to bind to its target.

Small molecule affinity ligands often comprise cyclical carbon or heterocyclic structures and/or aromatic or polyaromatic structures substituted with one or more of the above functional groups. Also of interest as small molecules are structures found among biomolecules, including peptides, saccharides, fatty acids, steroids, purines, pyrimidines, derivatives, structural analogs or combinations thereof. Such compounds may be screened to identify those of interest, where a variety of different screening protocols are known in the art.

The small molecule may be derived from a naturally occurring or synthetic compound that may be obtained from a wide variety of sources, including libraries of synthetic or natural compounds. For example, numerous means are available for random and directed synthesis of a wide variety of organic compounds and biomolecules, including the preparation of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known small molecules may be subjected to directed or random chemical modifications, such as acylation, alkylation, esterification, amidification, etc. to produce structural analogs.

As such, the small molecule may be obtained from a library of naturally occurring or synthetic molecules, including a library of compounds produced through combinatorial means, i.e. a compound diversity combinatorial library. When obtained from such libraries, the small molecule employed will have demonstrated some desirable affinity for the protein target in a convenient binding affinity assay. Combinatorial libraries, as well as methods for the production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

As pointed out, the affinity ligand can also be a large molecule. Of particular interest as large molecule affinity ligands are antibodies, as well as binding fragments and mimetics thereof. Where antibodies are the affinity ligand, they may be derived from polyclonal compositions, such that a heterogeneous population of antibodies differing by specificity are each tagged with the same tag nucleic acid, or monoclonal compositions, in which a homogeneous population of identical antibodies that have the same specificity for the target protein are each tagged with the same tag nucleic acid. As such, the affinity ligand may be either a monoclonal and polyclonal antibody. In yet other embodiments, the affinity ligand is an antibody binding fragment or mimetic, where these fragments and mimetics have the requisite binding affinity for the target protein. For example, antibody fragments, such as Fv, F(abN)₂ and Fab may be prepared by cleavage of the intact protein, e.g. by protease or chemical cleavage. Also of interest are recombinantly produced antibody fragments, such as single chain antibodies or scFvs, where such recombinantly produced antibody fragments retain the binding characteristics of the above antibodies. Such recombinantly produced antibody fragments generally include at least the V_{H} and V_{L} domains of the subject antibodies, so as to retain the binding characteristics of the subject antibodies. These recombinantly produced antibody fragments or mimetics of the subject invention may be readily prepared using any convenient methodology, such as the methodology disclosed in U.S. Pat. Nos. 5,851,829 and 5,965,371; the disclosures of which are herein incorporated by reference.

The above described antibodies, fragments and mimetics thereof may be obtained from commercial sources and/or prepared using any convenient technology, where methods of producing polyclonal antibodies, monoclonal antibodies, fragments and mimetics thereof, including recombinant derivatives thereof, are known to those of the skill in the art.

Importantly, the affinity ligand will be one that includes a domain or moiety that can be covalently attached to the nucleic acid tag without substantially abolishing the binding affinity for the affinity ligand to its target protein.

Tag Domain

The tag domain or component of the tagged affinity ligands is a nucleic acid that is sufficiently long to provide for hybridization under stringent conditions with its corresponding tag complement. As such, the length of the tag component generally ranges from about 10 to 70 nt in length, but is generally from about 18 to 60 and in many embodiments is from about 20 to 40 nucleotides in length. Generally, the tag component ranges in length from about 20 to 50 nt. The tag may be made up of ribonucleotides and deoxyribonucleotides as well as synthetic nucleotide residues that are capable of participating in Watson-Crick type or analogous base pair interactions.

The sequence of the tag nucleic acid is chosen or selected with respect to their complementary tag-complements, as described in greater detail infra. Once the sequence is identified, the tag nucleic acids may be synthesized using any convenient protocol, where representative protocols for synthesizing nucleic acids are described in greater detail infra in terms of the preparation of the tag complement or universal arrays employed in the subject methods.

Linking Moiety

The two components of the tagged affinity ligand conjugate are joined together either directly through a bond or indirectly through a linking group. Where linking groups are employed, such groups are chosen to provide for covalent attachment of the tag and affinity ligand moieties through the linking group, as well as maintain the desired binding affinity of the affinity ligand for its target protein. Linking groups of interest may vary widely depending on the affinity ligand moiety. The linking group, when present, should preferably be biologically inert. A variety of linking groups are known to those of skill in the art and find use in the subject conjugates. In many embodiments, the linking group is generally at least about 50 daltons, usually at least about 100 daltons and may be as large as 1000 daltons or larger, but generally will not exceed about 500 daltons and usually will not exceed about 300 daltons. Generally, such linkers will comprise a spacer group terminated at either end with a reactive functionality capable of covalently bonding to the drug or ligand moieties. Spacer groups of interest possibly include aliphatic and unsaturated hydrocarbon chains, spacers containing heteroatoms such as oxygen (ethers such as polyethylene glycol) or nitrogen (polyamines), peptides, carbohydrates, cyclic or acyclic systems that may possibly contain heteroatoms. Spacer groups may also be comprised of ligands that bind to metals such that the presence of a metal ion coordinates two or more ligands to form a complex. Specific spacer elements include: 1,4-diaminohexane, xylylenediamine, terephthalic acid, 3,6-dioxaoctanedioic acid, ethylenediamine-N,N-diacetic acid, 1,1′-ethylenebis(5-oxo-3-pyrrolidinecarboxylic acid), 4,4′-ethylenedipiperidine. Potential reactive functionalities include nucleophilic functional groups (amines, alcohols, thiols, hydrazides), electrophilic functional groups (aldehydes, esters, vinyl ketones, epoxides, isocyanates, maleimides), functional groups capable of cycloaddition reactions, forming disulfide bonds, or binding to metals. Specific examples include primary and secondary amines, hydroxamic acids, N-hydroxysuccinimidyl esters, N-hydroxysuccinimidyl carbonates, oxycarbonylimidazoles, nitrophenylesters, trifluoroethyl esters, glycidyl ethers, vinylsulfones, and maleimides. Specific linker groups that may find use in the subject tagged affinity ligands include heterofunctional compounds, such as azidobenzoyl hydrazide, N-[4-(p-azidosalicylamino)butyl]-3′-[2′-pyridyldithio]propionamid), bis-sulfosuccinimidyl suberate, dimethyladipimidate, disuccinimidyltartrate, N-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, and succinimidyl 4-[N-maleimidomethyl]cyclohexane-1-carboxylate, 3-(2-pyridyldithio)propionic acid N-hydroxysuccinimide ester (SPDP), 4-(N-maleimidomethyl)-cyclohexane-1-carboxylic acid N-hydroxysuccinimide ester (SMCC), and the like.

Preparation of Population of Tagged Affinity Ligands

The above described population of tagged target affinity ligands may be prepared using any convenient protocol. In many embodiments, tag nucleic acids will be conjugated to the affinity ligand, either directly or through a linking group. The components can be covalently bonded to one another through functional groups, as is known in the art, where such functional groups may be present on the components or introduced onto the components using one or more steps, e.g. oxidation reactions, reduction reactions, cleavage reactions and the like. Functional groups that may be used in covalently bonding the components together to produce the tagged affinity ligand include: hydroxy, sulfhydryl, amino, and the like. The particular portion of the different components that are modified to provide for covalent linkage will be chosen so as not to substantially adversely interfere with that components desired binding affinity for the target protein. Where necessary and/or desired, certain moieties on the components may be protected using blocking groups, as is known in the art, see, e.g. Green & Wuts, Protective Groups in Organic Synthesis (John Wiley & Sons) (1991). Methods for producing nucleic acid antibody conjugates are well known to those of skill in the art. See e.g. U.S. Pat. No. 5,733,523, the disclosure of which is herein incorporated by reference.

Tag Complement Arrays

As summarized above, another feature of the subject methods is that an array of tag complements, i.e. a universal array, is employed. The tag complement arrays of the subject invention have a plurality of probe spots stably associated with or immobilized on a surface of a solid support. A feature of the subject tag complement arrays is that at least a portion of the probe spots, and preferably substantially all of the probe spots, on the array are tag complement probe spots, where each tag complement probe spot is generally made up of a number or plurality of identical nucleic acid probe molecules that include a tag complement domain.

Probe Spots of the Arrays

As mentioned above, a feature of the subject invention is the nature of the probe spots, i.e. that at least a portion of, and usually substantially all of, the probe spots on the array are made up of probe nucleic acid compositions of tag complements, i.e. generally at least a substantial portion of the probe spots are tag complement probe spots. Each tag complement probe spot on the surface of the substrate is made up of tag complement nucleic acid probes, where the spot may be homogeneous with respect to the nature of the probe molecules present therein or heterogenous, e.g. as described in U.S. patent application Ser. No. 60/104,179, the disclosure of which is herein incorporated by reference.

A feature of the subject tag complement probe compositions is that they are made up of probe molecules that include a tag complement domain and a substrate surface binding domain. By tag complement domain is meant a stretch or region of nucleotides that has a sequence which is the complement of, i.e., is complementary to, a tag domain with which the subject array is used. In other words, the tag complement domain is a domain that hybridizes to a tag domain of a tagged affinity ligand acid during in the subject methods. The length of the tag complement domain may vary, but is, in many embodiments, substantially the same length as the tag domain to which it hybridizes during practice of the subject methods, where by substantially the same length is meant that the magnitude of any difference in lengths typically does not exceed about 15 nt and usually does not exceed about 10 nt. As such, the length of the subject tag complement domains generally ranges from about 10 to 70, usually from about 18 to 60 and more usually from about 20 to 40 nt. The sequence of nucleotides in the tag complement is chosen or selected based on a number of different parameters with respect to its corresponding tag, where these considerations and parameters are described in greater detail infra.

While in the broadest sense the probe molecules that make up the probe spots of the arrays employed in the subject methods may be any length, a feature of the probe compositions in the arrays employed in many of the embodiments of the subject invention is that the probe compositions are made up of long oligonucleotides. As such, the tag complement probes of the probe compositions range in length from about 50 to 150, typically from about 50 to 120 nt and more usually from about 60 to 100 nt, where in many preferred embodiments the probes range in length from about 65 to 85 nt. Such long oligonucleotides are further described in U.S. patent application Ser. No. 09/440,829, the disclosure of which is herein incorporated by reference.

In addition, the probe molecules of a given spot are chosen so that each tag complement probe molecule on the array is not homologous with any other distinct unique tag complement probe molecule present on the array, i.e. any other tag complement probe molecule on the array with a different base sequence. In other words, each distinct tag complement probe molecule of a probe composition corresponding to a first tag does not cross-hybridize with, or have the same sequence as, any other distinct unique tag complement probe molecule of any probe composition corresponding to a different target, i.e. an oligonucleotide of any other tag complement probe composition that is represented on the array. As such, nucleotide sequence of each unique tag complement probe molecule of a probe composition will have less than 90% homology, usually less than 70% homology, and more usually less than 50% homology with any other different tag complement probe molecule of a probe composition on the array corresponding to a different tag, where homology is determined by sequence analysis comparison using the FASTA program using default settings.

The tag complement probe molecules of each probe composition, or at least the tag complement portion of these molecules, are further characterized as follows. First, they have a GC content of from about 35% to 80%, usually between about 40 to 70%. Second, they have a substantial absence of: (a) secondary structures, e.g. regions of self-complementarity (e.g. hairpins), structures formed by intramolecular hybridization events; (b) long homopolymeric stretches, e.g. polyA stretches, such that in any give homopolymeric stretch, the number of contiguous identical nucleotide bases does not exceed 4; (c) long stretches (more than 8 nt) characterized by or enriched by the presence of repeating motifs, e.g GAGAGAGA, GAAGAGAA, etc.; (d) long stretches of homopurine or homopyrimidine rich (more than 8 nt) motifs; and the like.

The tag complement probes of the subject invention may be made up solely of the tag complement sequence as described above, e.g. sequence designed or present which is intended for hybridization to the probe's corresponding tag, or may be modified to include one or more non-tag complementary domains or regions, e.g. at one or both termini of the probe, where these domains may be present to serve a number of functions, including attachment to the substrate surface, to introduce a desired conformational structure into the probe sequence, etc.

One optional domain or region that may be present at one or more both termini of the long oligonucleotide probes of the subject arrays is a region enriched for the presence of thymidine bases, e.g. an oligo dT region, where the number of nucleotides in this region is typically at least 3, usually at least 5 and more usually at least 10, where the number of nucleotides in this region may be higher, but generally does not exceed about 25 and usually does not exceed about 20, where at least a substantial portion of, if not all of, the nucleotides in this region include a thymidine base, where by substantial portion is meant at least about 50, usually at least about 70 and more usually at least about 90 number % of all nucleotides in the oligo dT region. Certain probes of this embodiment of the subject invention, i.e. those in which the T enriched domain is an oligo dT domain, may be described by the following formula:

Tₙ-Nₘ-Tₖ;

wherein:

T is dTMP;

Nₘ is the target specific sequence of the probe in which N is either dTMP, dGMP, dCMP or dAMP and m is from 15 to 50; and

n and k are independently from 0 to 15, where when present n and/or k are preferably 5 to 10.

In yet other embodiments and often in addition to the above described T enriched domains, the subject probes may also include domains that impart a desired constrained structure to the probe, e.g. impart to the probe a structure which is fixed or has a restricted conformation. In many embodiments, the probes include domains which flank either end of the target specific domain and are capable of imparting a hairpin loop structure to the probe, whereby the target specific sequence is held in confined or limited conformation which enhances its binding properties with respect to its corresponding target during use. In these embodiments, the probe may be described by the following formula:

Tₙ-Nₚ-Nₘ-Nₒ-Tₖ

wherein:

T is dTMP;

N is dTMP, dGMP, dCMP or dAMP;

m is an integer from 15 to 50;

n and k are independently from 0 to 15, where when present n and/or k are preferably 5 to 10, where in many embodiments k=n=5 to 10, more preferably 10; and

p and o are independently 5 to 20, usually 5 to 15, and more usually about 10, wherein in many embodiments p=o=5 to 15 and preferably 10;

such that Nₘ is the target specific sequence; and

Nₒ and Nₚ are self complementary sequences, e.g. they are complementary to each other, such that under hybridizing conditions the probe forms a hairpin loop structure in which the stem is made up of the Nₒ and Nₚ sequences and the loop is made up of the target specific sequence, i.e. Nₘ.

The tag complement probe compositions that make up each tag complement probe spot on the array will be substantially, usually completely, free of non-nucleic acids, i.e. the probe compositions will not include or be made up of non-nucleic acid biomolecules found in cells, such as proteins, lipids, and polysaccharides. In other words, the oligonucleotide spots of the arrays are substantially, if not entirely, free of non-nucleic acid cellular constituents.

The tag complement probes may be nucleic acid, e.g. RNA, DNA, or nucleic acid mimetics, e.g. nucleic acids that differ from naturally occurring nucleic acids in some manner, e.g. through modified backbones, sugar residues, bases, etc., such as nucleic acids comprising non-naturally occurring heterocyclic nitrogenous bases, peptide-nucleic acids, locked nucleic acids (see Singh & Wengel, Chem. Commun. (1998) 1247-1248); and the like. In many embodiments, however, the nucleic acids are not modified with a functionality which is necessary for attachment to the substrate surface of the array, e.g. an amino functionality, biotin, etc.

The tag complement probe spots made up of the tag complement probes as described above and present on the array may be any convenient shape, but will typically be circular, elliptoid, oval or some other analogously curved shape. The total amount or mass of tag complement probe molecules present in each spot will be sufficient to provide for adequate hybridization and detection of tagged affinity ligand during the assay in which the array is employed. Generally, the total mass of nucleic acids in each spot will be at least about 0.1 ng, usually at least about 0.5 ng and more usually at least about 1 ng, where the total mass may be as high as 100 ng or higher, but will usually not exceed about 20 ng and more usually will not exceed about 10 ng. The copy number of all of the oligonucleotides in a spot will be sufficient to provide enough hybridization sites for tagged target molecule to yield a detectable signal, and will generally range from about 0.001 fmol to 10 fmol, usually from about 0.005 fmol to 5 fmol and more usually from about 0.01 fmol to 1 fmol. Where the spot is made up of two or more distinct tag complement probe molecules of differing sequence, the molar ratio or copy number ratio of different oligonucleotides within each spot may be about equal or may be different, wherein when the ratio of unique nucleic acids within each spot differs, the magnitude of the difference will usually be at least 2 to 5 fold but will generally not exceed about 10 fold.

Where the spot has an overall circular dimension, the diameter of the spot will generally range from about 10 to 5,000 μm, usually from about 20 to 1,000 μm and more usually from about 50 to 500 μm. The surface area of each spot is at least about 100 μm², usually at least about 200 μm² and more usually at least about 400 μm², and may be as great as 25 mm² or greater, but will generally not exceed about 5 mm², and usually will not exceed about 1 mm².

Additional Array Features

The arrays of the subject invention are characterized by having a plurality of probe spots as described above stably associated with the surface of a solid support. The density of probe spots on the array, as well as the overall density of probe and non-probe nucleic acid spots (where the latter are described in greater detail infra) may vary greatly. As used herein, the term nucleic acid spot refers to any spot on the array surface that is made up of nucleic acids, and as such includes both probe nucleic acid spots and non-probe nucleic acid spots. The density of the nucleic acid spots on the solid surface is at least about 5/cm² and usually at least about 10/cm² and may be as high as 1000/cm² or higher, but in many embodiments does not exceed about 1000/cm², and in these embodiments usually does not exceed about 500/cm² or 400/cm², and in certain embodiments does not exceed about 300/cm². The spots may be arranged in a spatially defined and physically addressable manner, in any convenient pattern across or over the surface of the array, such as in rows and columns so as to form a grid, in a circular pattern, and the like, where generally the pattern of spots will be present in the form of a grid across the surface of the solid support.

In the subject arrays, the spots of the pattern are stably associated with or immobilized on the surface of a solid support, where the support may be a flexible or rigid support. By “stably associated” it is meant that the oligonucleotides of the spots maintain their position relative to the solid support under hybridization and washing conditions. As such, the oligonucleotide members which make up the spots can be non-covalently or covalently stably associated with the support surface based on technologies well known to those of skill in the art. Examples of non-covalent association include nonspecific adsorption, binding based on electrostatic (e.g. ion, ion pair interactions), hydrophobic interactions, hydrogen bonding interactions, specific binding through a specific binding pair member covalently attached to the support surface, and the like. Examples of covalent binding include covalent bonds formed between the spot oligonucleotides and a functional group present on the surface of the rigid support, e.g. —OH, where the functional group may be naturally occurring or present as a member of an introduced linking group. In many preferred embodiments, the nucleic acids making up the spots on the array surface, or at least the tag complement molecules of the probe spots, are covalently bound to the support surface, e.g. through covalent linkages formed between moieties present on the probes (e.g. thymidine bases) and the substrate surface, etc.

As mentioned above, the array is present on either a flexible or rigid substrate. By flexible is meant that the support is capable of being bent, folded or similarly manipulated without breakage. Examples of solid materials which are flexible solid supports with respect to the present invention include membranes, flexible plastic films, and the like. By rigid is meant that the support is solid and does not readily bend, i.e. the support is not flexible. As such, the rigid substrates of the subject arrays are sufficient to provide physical support and structure to the polymeric targets present thereon under the assay conditions in which the array is employed, particularly under high throughput handling conditions. Furthermore, when the rigid supports of the subject invention are bent, they are prone to breakage.

The solid supports upon which the subject patterns of spots are presented in the subject arrays may take a variety of configurations ranging from simple to complex, depending on the intended use of the array. Thus, the substrate could have an overall slide or plate configuration, such as a rectangular or disc configuration. In many embodiments, the substrate will have a rectangular cross-sectional shape, having a length of from about 10 mm to 200 mm, usually from about 40 to 150 mm and more usually from about 75 to 125 mm and a width of from about 10 mm to 200 mm, usually from about 20 mm to 120 mm and more usually from about 25 to 80 mm, and a thickness of from about 0.01 mm to 5.0 mm, usually from about 0.01 mm to 2 mm and more usually from about 0.01 to 1 mm. Thus, in one representative embodiment the support may have a micro-titre plate format, having dimensions of approximately 125×85 mm. In another representative embodiment, the support may be a standard microscope slide with dimensions of from about 25×75 mm.

The substrates of the subject arrays may be fabricated from a variety of materials. The materials from which the substrate is fabricated should ideally exhibit a low level of non-specific binding during hybridization events. In many situations, it will also be preferable to employ a material that is transparent to visible and/or UV light. For flexible substrates, materials of interest include: nylon, both modified and unmodified, nitrocellulose, polypropylene, and the like, where a nylon membrane, as well as derivatives thereof, is of particular interest in this embodiment. For rigid substrates, specific materials of interest include: glass; plastics, e.g. polytetrafluoroethylene, polypropylene, polystyrene, polycarbonate, and blends thereof, and the like; metals, e.g. gold, platinum, and the like; etc. Also of interest are composite materials, such as glass or plastic coated with a membrane, e.g. nylon or nitrocellulose, etc.

The substrates of the subject arrays comprise at least one surface on which the pattern of spots is present, where the surface may be smooth or substantially planar, or have irregularities, such as depressions or elevations. The surface on which the pattern of spots is present may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like. Polymeric layers of interest include layers of: peptides, proteins, polynucleic acids or mimetics thereof, e.g. peptide nucleic acids and the like; polysaccharides, phospholipids, polyurethanes, polyesters, polycarbonates, polyureas, polyamides, polyethyleneamines, polyarylene sulfides, polysiloxanes, polyimides, polyacetates, polyacrylamides, and the like, where the polymers may be hetero- or homopolymeric, and may or may not have separate functional moieties attached thereto, e.g. conjugated.

The total number of spots on the substrate will vary depending on the number of different oligonucleotide probe spots (oligonucleotide probe compositions) one wishes to display on the surface, as well as the number of non probe spots, e.g control spots, orientation spots, calibrating spots and the like, as may be desired depending on the particular application in which the subject arrays are to be employed. Generally, the pattern present on the surface of the array will comprise at least about 10 distinct nucleic acid spots, usually at least about 20 nucleic acid spots, and more usually at least about 50 nucleic acid spots, where the number of nucleic acid spots may be as high as 10,000 or higher, but will usually not exceed about 5,000 nucleic acid spots, and more usually will not exceed about 3,000 nucleic acid spots and in many instances will not exceed about 2,000 nucleic acid spots. In certain embodiments, it is preferable to have each distinct probe spot or probe composition be presented in duplicate, i.e. so that there are two duplicate probe spots displayed on the array for a given target. In certain embodiments, each target represented on the array surface is only represented by a single type of oligonucleotide probe. In other words, all of the oligonucleotide probes on the array for a give target represented thereon have the same sequence. In certain embodiments, the number of spots will range from about 200 to 1200. The number of tag complement probe spots present in the array will typically make up a substantial proportion of the total number of nucleic acid spots on the array, where in many embodiments the number of probe spots is at least about 50 number %, usually at least about 80 number % and more usually at least about 90 number % of the total number of nucleic acid spots on the array. As such, in many embodiments the total number of tag complement probe spots on the array ranges from about 50 to 20,000, usually from about 100 to 10,000 and more usually from about 200 to 5,000.

In the arrays of the subject invention (particularly those designed for use in high throughput applications, such as high throughput analysis applications), a single pattern of tag complement spots may be present on the array or the array may comprise a plurality of different tag complement spot patterns, each pattern being as defined above. When a plurality of different tag complement spot patterns are present, the patterns may be identical to each other, such that the array comprises two or more identical tag complement spot patterns on its surface, or the oligonucleotide spot patterns may be different, e.g. in arrays that have two or more different sets of tag complements probes present on their surface, e.g an array that has a pattern of tag complement spots corresponding to first population of tags and a second pattern of tag complement spots corresponding to a second population of tags. Where a plurality of tag complement spot patterns are present on the array, the number of different tag complement spot patterns is at least 2, usually at least 6, more usually at least 24 or 96, where the number of different patterns will generally not exceed about 384.

Where the array comprises a plurality of tag complement spot patterns on its surface, preferably the array comprises a plurality of reaction chambers, wherein each chamber has a bottom surface having associated therewith an pattern of tag complement spots and at least one wall, usually a plurality of walls surrounding the bottom surface. See e.g. U.S. Pat. No. 5,545,531, the disclosure of which is herein incorporated by reference. Of particular interest in many embodiments are arrays in which the same pattern of spots in reproduced in 24 or 96 different reaction chambers across the surface of the array.

Within any given pattern of spots on the array, there may be a single tag complement spot that corresponds to a given tag or a number of different tag complement spots that correspond to the same tag, where when a plurality of different tag complement spots are present that correspond to the same tag, the tag complement probe compositions of each spot that corresponds to the same tag may be identical or different. In other words, a plurality of different tags are represented in the pattern of tag complement spots, where each tag may correspond to a single tag complement spot or a plurality of spots, where the tag complement probe compositions among the plurality of spots corresponding to the same tag may be the same or different. Where a plurality of spots (of the same or different composition) corresponding to the same tag is present on the array, the number of spots in this plurality will be at least about 2 and may be as high as 10, but will usually not exceed about 5. As mentioned above, however, in many preferred embodiments, any given tag is represented by only a single type of tag complement probe spot, which may be present only once or multiple times on the array surface, e.g. in duplicate, triplicate etc.

The number of different tag complements present on the array, and therefore the number of different tags represented on the array, is at least about 2, usually at least about 10 and more usually at least about 20, where in many embodiments the number of different tags represented on the array is at least about 50 and more usually at least about 100. The number of different tags represented on the array may be as high as 5,000 or higher, but in many embodiments will usually not exceed about 3,000 and more usually will not exceed about 2,500. A tag is considered to be represented on an array if it is able to hybridize to one or more tag complement probe compositions on the array.

Additional Features of the Tag-Tag Complement Pairs

The tags and tag complements of the tagged affinity labels and arrays, respectively, employed in any given embodiment of subject methods are, in many embodiments, characterized by the following additional features. In many embodiments of the subject invention, any tag or tag complement that is employed is a member of a collection of tag-tag complement pairs in which the hybridization efficiency of each constituent tag-tag complement pair is substantially the same, i.e. all of the tag-tag complement pairs in the population or collection of tag-tag complement pairs are characterized by having substantially the same hybridization efficiency. As such, the hybridization of a tag to its complementary tag complement in any given tag-tag complement pair of the population or collection is substantially the same as that observed for any other given tag-tag complement pair in the population. By substantially the same is meant that the hybridization efficiency is the same or, if it varies, it does not vary by more than about 10 fold, usually by more than about 5 fold and more usually by more than about 3 fold. Hybridization or binding efficiency refers to the ability of the tag complement to bind to its tag under the hybridization conditions in which the array is used. Put another way, binding efficiency refers to the duplex yield obtainable with a given tag complement and its complementary tag after performing a hybridization experiment. In addition to having substantially the same hybridization or binding efficiency, the tag-tag complement pairs are typically further characterized by exhibiting high binding efficiency. In many embodiments, the tag-tag complement pairs present in the population or collection employed in the subject methods exhibit high hybridization efficiency having a binding efficiency of 0.1%, usually at least 0.5% and more usually at least 2% binding of tagget affinity ligands present in the hybridization assay with the tag complement probe arrays of the invention.

In addition to exhibiting substantially the same high hybridization efficiency, the tag-tag complement pairs of the collections employed in the subject methods are further chosen to provide for low levels of cross hybridization, i.e. low levels of non-specific hybridization or binding. In other words, the sequence of the tag complement and its corresponding (e.g. complementary) tag are chosen to provide for low non-specific hybridization or non-specific binding, i.e. unwanted cross-hybridization, under stringent conditions. A given tag is considered to be substantially non-complementary to a given tag complement if the tag has homology to the tag complement of less than 60%, more commonly less than 50% and most commonly less than 40%, as determined using the FASTA program with default settings. In certain embodiments, tag-tag complement pairs having low non-specific hybridization characteristics and finding use in the subject methods are those in which the relative ability of the tag or tag complement to hybridize to a non-complementary nucleic acid, i.e., other tag complements or tags for which they are not substantially complementary, is less than 10%, usually less than 5 or 2% and preferably less than 1% of their ability to bind to their complementary nucleic acid, i.e. tag or tag complement. For example, in a side-by-side hybridization assay, tag complements having low non-specific hybridization characteristics are those which generate a positive signal, if any, when contacted with a tag composition that does not include a complementary tag for the tag complement, that is less than about 10%, usually least than about 3 or 2% and more usually less than about 1% of the signal that is generated by the same tag complement when it is contacted with a tag composition that includes a complementary tag.

The sequences of the individual tags and tag complements that make up the population of tag-tag complement pairs employed in the subject methods and having the characteristics described above may be determined using any convenient protocol.

In many embodiments, the protocol that is employed identifies sequences that meet the following parameters or criteria. First, the sequence that is chosen as the tag or tag complement sequence should yield a tag-tag complement pair the members of which, i.e. the tag or tag complement, do not cross-hybridize with, or are not homologous to, the members of any other tag-tag complement pair in the collection or population of pairs that is employed. Second, the sequence that is chosen for a given member of a tag-tag complement pair in the population should be chosen such that that member has a low homology to a nucleotide sequence found in any known gene, e.g. any gene whose sequence has been deposited in an accessible electronic database. As such, sequences that are avoided include those found in: highly expressed gene products, structural RNAs, repetitive sequences found in the RNA sample to be tested with the array and sequences found in vectors. A further consideration is to select sequences which provide for minimal or no secondary structure, structure which allows for optimal hybridization but low nonspecific binding, equal or similar thermal stabilities, and optimal hybridization characteristics. A final consideration is to select sequences that give rise to tag-tag complement pairs that show similar high binding efficiency and low cross-hybridization, as described above. Finally, the sequences of the members of the tag-tag complement constituent members of the population are chosen such that they exhibit substantially the same hybridization efficiency, where the difference in hybridization efficiency between any two tag-tag complement pairs in the population preferably does not exceed about 10 fold, more preferably does not exceed about 5 fold and most preferably does not exceed about 3 fold.

One representative protocol for identifying the sequence of the tags and tag complements that make up the subject populations of tag-tag complement pairs is as follows. First the general length of the tag and tag complements is identified. Generally, the length of tag and tag complements ranges from about 10 to 50, usually from about 20 to 25 and more usually from about 20 to 35 nt. In a given collection, the tag and tag complements may be the same length or of different length, where when there is variation in lengths, the variation is not substantial, such that any difference in length does not exceed about 20, usually does not exceed about 10 and more usually does not exceed about 7 or even 5 nt.

Once a tag/tag complement length is identified, all possible sequences for that length are then determined. For example, where the length is 25 nt and the tags/tag complements are to be polymers of the four naturally occurring dideoxynucleotides, a total of 4²⁵ sequences are possible. Generally, these sequence are conveniently determined using a computational means. This initial population of potential sequence is then subjected to the following initial selection or screening steps. In other words, screening criteria are employed for this initial population to exclude non-optimal sequences, where sequences that are excluded or screened out in this step include: (a) those with strong secondary structure or self-complementarity (for example long hairpins); (b) those with very high (more than 70%) or very low (less than 40%) GC content; (c) those with long stretches (more than 4) of identical consecutive bases or long stretches (more than 8 nt) of sequences enriched in some bases, purine or pyrimidine stretches or particular motifs, like GAGAGAGA, GAAGAGAA; and the like. This step results in a reduction in the population of candidate sequences.

In the next step, sequences are selected that have similar melting temperatures or thermodynamic stability which will provide similar performance in hybridization assays with tag nucleic acids. Of interest is the identification of probes that can participate in duplexes whose differences in melting temperature does not exceed about 15, usually at 10 and more usually 5° C.

Next, the sequence of all sequences deposited in GenBank are searched in order to select tag/tag complements sequences that are unique and are not homologous to any entry in GenBank, particularly any entry related to phage, viral, prokaryotic, archaebacteria, eukaryotic. A unique sequence is defined as a sequence which at least does not have significant homology to any other sequence on the array. For example, where one is interested in identifying suitable 30 base long tag complement probes, sequences which do not have homology of more than about 80% to any consecutive 30 base segment of any other potential target sequences are selected. This step typically results in a reduced population of candidate sequences as compared to the initial population of possible sequences identified for each specific target.

The final step in this representative design process is to select from the remaining sequences those sequences which provide for low levels of non-specific hybridization and similar high efficiency hybridization, as described above. This final selection is accomplished by practicing the following steps:

For each potential sequence, a tag complement is synthesized and covalently attached (in similar amount) to a solid surface, thus generating array of tag complements;

A set of control labeled tags is then synthesized and combined, where each of the control tags in the set is present in substantially the same amount as the other control tags. The number of different labeled tags in the control set is usually less than the number of tag complements in the array. Usually the set of control tags is about 50%, more commonly 80% and most commonly 90% from the number of tag complements in the array.

The set of control tags is then hybridized with the tag complement array and hybridization signals for all tag complements are detected. Intensities of signal for tag complements which have labeled complementary tags in hybridization solution (i.e. in the control tag set) reflect efficiency and differences in efficiency of different tags. For the tag complements which do not have complementary tag sequences in the control set, the intensity of hybridization signals reflects the level of non-specific hybridization.

The above steps are then repeated with another set of control tags in order to obtain comprehensive information concerning hybridization efficiency and level of non-specific hybridization for each tag complement in the array.

Using information obtained from the above steps, tag-tag complement pairs are then selected which satisfy the following criteria:

Differences in hybridization efficiency between all selected tag-tag complement pairs in the array are less than 10-fold, more commonly less than 5-fold and most commonly less than 3-fold.

Any tag-tag complement pairs which show level of cross hybridization (non specific hybridization) more than 10%, more commonly 2% and most commonly more than 1% from level of tag-specific hybridization were rejected for further use for the purpose of invention.

The above protocol identifies a set of tag-tag complement pairs that can be employed in the subject methods from an initial set or collection of possible pairs based on the desired length of the tag/tag complement pairs. For example, where one initially has a total of 4²⁵ potential sequences and tag-tag complement pairs to choose from, the above protocol allows one to select about 20,000, commonly about 10,000 and more commonly about 5,000 different tag-tag complement pairs, where the identified and selected pairs exhibit similar very efficient hybridization characteristics and minimal levels of non-specific hybridization. The above protocols also provide a number of additional advantages, including: (a) significantly eliminating the need for using theoretical and non-reliable algorithms for tag selection; (b) significantly improving the quality of expression data generated by universal array; (c) simplify data analysis: and (d) significantly reducing the cost of array production.

Non-Tag Complement Probe Spots

In addition to the tag complement spots comprising the tag complement probe compositions (i.e. tag probe spots), the subject arrays may comprise one or more additional nucleic acid spots which do not correspond to tag nucleic acids. In other words, the array may comprise one or more non-probe nucleic acid spots, e.g., orientation spots may also be included on the array, where such spots serve to simplify image analysis of hybrid patterns, spots for calibration or quantitative standards, and the like. These latter types of spots are distinguished from the tag complement probe spots, i.e. they are non-probe spots.

Array Preparation

The subject arrays can be prepared using any convenient means. One means of preparing the subject arrays is to first synthesize the nucleic acids for each spot and then deposit the nucleic acids as a spot on the support surface. The nucleic acids may be prepared using any convenient methodology, where chemical synthesis procedures using phorphoramidite or analogous protocols in which individual bases are added sequentially without the use of a polymerase, e.g. such as is found in automated solid phase synthesis protocols, and the like, are of particular interest, where such techniques are well known to those of skill in the art.

Following synthesis of the subject tag complement probe molecules, the probes are stably associated with the surface of the solid support. This portion of the preparation process typically involves deposition of the probes, e.g. a solution of the probes, onto the surface of the substrate, where the deposition process may or may not be coupled with a covalent attachment step, depending on how the probes are to be stably attached to the substrate surface, e.g. via electrostatic interactions, covalent bonds, etc. The prepared oligonucleotides may be spotted on the support using any convenient methodology, including manual techniques, e.g. by micro pipette, ink jet, pins, etc., and automated protocols. Of particular interest is the use of an automated spotting device, such as the BioGrid Arrayer (Biorobotics).

Where desired, the tag complement molecules can be covalently bonded to the substrate surface using a number of different protocols. For example, functionally active groups such as amino, etc., can be introduced onto the 5′ or 3′ ends of the oligonucleotides, where the introduced functionalities are then reacted with active surface groups on the substrate to provide the covalent linkage. In certain preferred embodiments, the probes are covalently bonded to the surface of the substrate using the following protocol. In this process, the probes are covalently attached to the substrate surface under denaturing conditions. Typically, a denaturing composition of each probe is prepared and then deposited on the substrate surface. By denaturing composition is meant that the probe molecules present in the composition are not participating in secondary structures, e.g. through self-hybridization or hybridization to other molecules in the composition. The denaturing composition, typically a fluid composition, may be any composition which inhibits the formation of hydrogen bonds between complementary nucleotide bases. Thus, compositions of interest are those that include a denaturing agent, e.g. urea, formamide, sodium thiocyanate, etc., as well as solutions having a high pH, e.g. 12 to 13.5, usually 12.5 to 13, or a low pH, e.g. 1 to 4, usually 1 to 3; and the like. In many preferred embodiments, the composition is a strongly alkaline solution of the long oligonucleotide, where the composition comprises a base, e.g. sodium hydroxide, lithium hydroxide, potassium hydroxide, ammonium hydroxide, tetramethyl ammonium hydroxide, ammonium hydroxide, etc, in sufficient amounts to impart the desired high pH to the composition, e.g. 12.5 to 13.0. In other embodiments, high salt concentrations, e.g., 0.5 to 2 M LiCl, 2×SSC, 0.5 to 1.0 M NaHCO₃, etc., and/or detergents, e.g., 0.01 to 0.1% SDS, etc., may be employed. The concentration of long oligonucleotide in the composition typically ranges from about 0.1 to 10 μM, usually from about 0.5 to 5 μM. In yet other embodiments, deposition is under non-denaturing conditions. Following deposition of the denaturing composition of the long oligonucleoide probe onto the substrate surface, the deposited probe is exposed to UV radiation of sufficient wavelength, e.g. from 250 to 350 nm, to cross link the deposited probe to the surface of the substrate. The irradiation wavelength for this process typically ranges from about 50 to 1000 mjoules, usually from about 100 to 500 mjoules, where the duration of the exposure typically lasts from about 20 to 600 sec, usually from about 30 to 120 sec.

The above protocol for covalent attachment results in the random covalent binding of the probe to the substrate surface by one or more attachment sites on the probe, where such attachment may optionally be enhanced through inclusion of oligo dT regions at one or more ends of the probes, as discussed supra. An important feature of the above process is that reactive moieties, e.g. amino, that are not present on naturally occurring probes are not employed in the subject methods. As such, the subject methods are suitable for use with probes that do not include moieties that are not present on naturally occurring nucleic acids.

The above described covalent attachment protocol may be used with a variety of different types of substrates. Thus, the above described protocols can be employed with solid supports, such as glass, plastics, membranes, e.g. nylon, and the like. The surfaces may or may not be modified. For example, the nylon surface may be charge neutral or positively charged, where such substrates are available from a number of commercial sources. For glass surfaces, in many embodiments the glass surface is modified, e.g. to display reactive functionalities, such as amino, phenyl isothiocyanate, etc.

Contacting Universal Array with Tagged Affinity Ligands

As summarized above, the subject methods are methods of detecting the presence of one or more analytes, e.g. proteins, in a sample. In practicing the subject methods, one or more binding complexes is produced on the surface of a tag complement or universal array, where the one or more surface bound binding complexes are then detected and related to the presence of the analyte in the sample. A feature of the subject methods is that a hybridization step is employed, in which tagged affinity ligands are contacted with a tag complement array, i.e. a universal array of tag complements, under hybridization conditions. Depending on the particular protocol that is employed, the tagged affinity ligands may or may not be bound to their target analyte or binding pair member, e.g. protein, when they are contacted with the array under hybridization conditions. As such, in one embodiment of the subject invention, a universal array is contacted with a population or set of tagged affinity ligands under hybridization conditions, where the affinity ligands have not yet been contacted with the sample to be assayed. As such, hybridization occurs between complementary surface bound tag complements and solution phase tagged affinity ligands to produce an array of surface bound affinity ligands. The array of surface bound affinity ligands is the contacted with the sample to produce the surface bound binding complexes that are detected and related to the presence of the target analyte(s) in the sample. In yet other embodiments, a population of distinct tagged affinity ligands is first contacted with the sample to be assayed to produce a population of solution phase tagged affinity ligand/analyte complexes. These solution phase complexes are then contacted with the array under hybridization conditions and any resultant surface bound binding complexes that include the analyte are detected and related to the presence of analyte in the sample. This latter format is preferred in many embodiments of the subject invention. As such, this latter format is now described in greater detail below, where modifications to the below described protocol may be readily made by those of skill in the art in order to practice the former embodiment.

As mentioned above, in a preferred embodiment a population of distinct tagged affinity ligands is contacted with a sample to be assayed under conditions sufficient for binding to occur between any affinity ligand and its target analyte, e.g. protein, if present in the sample. The number of distinct tagged affinity ligands in the population that is contacted with the sample is generally at least about 10, usually at least about 20 and more usually at least about 50, where in many embodiments the number of different affinity ligands is at least 75, usually at least 100 and often may be much greater. In many embodiments, the number of distinct tagged affinity ligands does not exceed about 5,000, usually does not exceed about 3,000 and more usually does not exceed about 2,000.

The sample with which the population of tagged affinity ligands is contacted may be any sample of interest to be assayed, but in many embodiments is a physiological sample. Where the sample is a physiological sample, the sample is generally obtained from a physiological source. The physiological source is often eukaryotic, with physiological sources of interest including sources derived from single celled organisms such as yeast and multicellular organisms, including plants and animals, particularly mammals, where the physiological sources from multicellular organisms may be derived from particular organs or tissues of the multicellular organism, or from isolated cells derived therefrom. In certain embodiments one is interested in assaying, testing or evaluating two related physiological sources. Thus, the physiological sources may be different cells from different organisms of the same species, e.g. cells derived from different humans, or cells derived from the same human (or identical twins) such that the cells share a common genome, where such cells will usually be from different tissue types, including normal and diseased tissue types, e.g. neoplastic, cell types. In obtaining the sample to be analyzed from the physiological source from which it is derived, the physiological source may be subjected to a number of different processing steps, where such processing steps might include tissue homogenization, nucleic acid extraction and the like, where such processing steps are known to the those of skill in the art.

Once the sample is prepared, the sample is contacted with the population of tagged affinity ligands under conditions sufficient for binding to occur between affinity ligands and their target analytes, if present in the sample. Conditions sufficient for binding to occur may be readily determined by those of skill in the art, e.g. physiological conditions may be employed (such as a temperature ranging from about 30 to 40, usually from about 35 to 40° C. and a pH ranging from about 6 to 8, usually from about 6.5 to 7.5). Contact is achieved using any convenient protocol, e.g. mixing, etc. Following the contact, the resultant mixture is generally maintained for a sufficient period of time for binding complexes to be produced between affinity ligands and their specific binding member pairs present in the sample. The solution phase binding complexes produced in this step are made up of the tagged affinity ligands bound to target analytes, e.g. target proteins. For example, tagged affinity ligand/target protein binding complexes are the product of this step when the target analyte is a protein.

Following production of the solution phase binding complexes, the next step is to contact the solution phase binding complexes with a universal array of tag complements under hybridization conditions sufficient to produce surface bound binding complexes. In this step, the hybridization conditions can be adjusted, as desired, to provide for an optimum level of specificity in view of the particular assay being performed. Suitable hybridization conditions are well known to those of skill in the art and reviewed in Maniatis et al, supra and WO 95/21944. Of particular interest in many embodiments is the use of stringent conditions during hybridization, i.e. conditions that are optimal in terms of rate, yield and stability for specific tag-tag complement hybridization and provide for a minimum of non-specific tag-tag complement interaction. Stringent conditions are known to those of skill in the art. In the present invention, stringent conditions are typically characterized by temperatures ranging from 15 to 35, usually 20 to 30° C. less than the melting temperature of the tag-tag complement duplexes, which melting temperature is dependent on a number of parameters, e.g. temperature, buffer compositions, size of probes and targets, concentration of probes and targets, etc. As such, the temperature of hybridization typically ranges from about 55 to 70, usually from about 60 to 68° C. In the presence of denaturing agents, the temperature may range from about 35 to 45, usually from about 37 to 42° C. The stringent hybridization conditions are further typically characterized by the presence of a hybridization buffer, where the buffer is characterized by one or more of the following characteristics: (a) having a high salt concentration, e.g. 3 to 6×SSC (or other salts with similar concentrations); (b) the presence of detergents, like SDS (from 0.1 to 20%), triton X100 (from 0.01 to 1%), monidet NP40 (from 0.1 to 5%) etc.; (c) other additives, like EDTA (typically from 0.1 to 1 μM), tetramethylammonium chloride; (d) accelerating agents, e.g. PEG, dextran sulfate (5 to 10%), CTAB, SDS and the like; (e) denaturing agents, e.g. formamide, urea etc.; and the like.

The above hybridization step results in the production of surface bound binding complexes, where the surface bound binding complexes are made up of the tag of a tagged affinity ligand hybridized to a surface bound tag complement and the affinity ligand of the tagged affinity ligand bound to its target analyte, e.g. protein. As used herein, the term “surface bound binding complex” does not include affinity ligands hybridized to a tag complement that are not also bound to their target protein. The presence of the resultant surface bound complexes from the hybridization step are detected using any convenient detection protocol. Many different protocols for detecting the presence of surface bound binding complexes are known to those of skill in the art, where the detection method may be qualitative or quantitative depending on the particular application in which the subject method is being performed, where the particular detection protocol employed may or may not use a detectable label. Representative detection protocols that may be employed include those described in WO 00/04389 and WO 00/04382; the disclosures of which are herein incorporated by reference. Representative non-label protocols include surface plasmon resonance, total internal reflection, Brewster Angle microscopy, optical waveguide light mode spectroscopy, surface charge elements, ellipsitometry, etc., as described in U.S. Pat. No. 5,313,264, the disclosure of which is herein incorporated by reference. Alternatively, detectable label based protocols, including protocols that employ a signal producing system, may be employed. Examples of directly detectable labels include isotopic and fluorescent moieties. Isotopic moieties or labels of interest include ³²P, ³³P, ³⁵S, ¹²⁵I, and the like. Fluorescent moieties or labels of interest include coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. texas red, tetramethylrhodamine, eosins and erythrosins, cyanine dyes, e.g. Cy3 and Cy5, macrocyclic chelates of lanthanide ions, e.g. quantum dye, fluorescent energy transfer dyes, such as thiazole orange-ethidium heterodimer, TOTAB, etc. Labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugate antibody; and the like. Depending on the particular protocol employed, the label may be incorporated into the that target analyte or protein, incorporated into the tagged affinity label, or present on a separate reactant that is employed in the detection step. See e.g. WO 00/004389, the disclosure of which is herein incorporated by reference.

Depending on the particular detection protocol employed, the assay may further include a separation step prior to the above discussed hybridization step, where in the separation step solution phase binding complexes made up of tagged affinity ligands bound to their corresponding target analytes are separated from tagged affinity ligands that are not bound to a target analyte. Any convenient separation protocol may be employed, where in many embodiments the separation protocol will be one based on size, e.g. electrophoretic separation, column chromatography, density based separation, etc.

Following detection of the surface bound binding complexes, the presence of any surface bound binding complexes is then related to the presence of the one or more analytes in the sample. This relating step is readily accomplished in that the position on the array at which a particular surface bound complex is located indicates the identify of the analyte or protein, since the affinity ligand for the protein is attached to a known specific tag that in turn hybridizes to a known location on the array. Thus, this relating step merely comprises determining the location on the array on which a binding complex is present, comparing that location to a reference that provides information regarding the correlation of each location to a particular analyte and thereby deriving the identity of the analyte in the sample. In sum, the location of the surface bound binding complexes is used to determine the identity of the one or more analytes of interest in the sample.

In certain embodiments, as mentioned above, two or more physiological sources are assayed according to the above protocols in order to generated analyte profiles for the two or more sources that may be compared. In such embodiments, each population of tagged affinity ligands may be separately contacted to identical universal arrays or together to the same array under conditions of hybridization, preferably under stringent hybridization conditions, depending on whether a means for distinguishing the patterns generated by the different populations is employed, e.g. distinguishable labels, such as two or more different emission wavelength fluorescent dyes, like Cy3 and Cy5, two or more isotopes with different energy of emission, like ³²P and ³³P, gold or silver particles with different scattering spectra, labels which generate signals under different treatment conditions, like temperature, pH, treatment by additional chemical agents, etc., or generate signals at different time points after treatment.

By way of further illustration, the following representative protein assay is summarized. Where one is interested in assaying a sample for the presence of 100 different proteins, a collection of 100 different tagged affinity ligands is prepared, where each different affinity ligand in the collection specifically binds to a different protein member of the 100 different proteins being assayed. The collection of 100 different tagged affinity ligands, e.g. nucleic acid tagged monoclonal antibodies, is then contacted with the sample being assayed under conditions sufficient for binding complexes to be produced between the tagged affinity ligands and their corresponding target proteins in the sample. Any resultant binding complexes in the sample are then separated from the remaining tagged affinity ligands. The isolated binding complexes are then hybridized to a universal array of tag complements and the resultant surface bound binding complexes are detected and the location of the detected binding complexes is used to determine which of the 100 proteins of interest is present in the sample.

Utility

The subject methods find use in a variety of different applications, where representative applications of interest include analyte detection, drug development, toxicity testing, clinical diagnostics, etc., where representative uses for the subject methods and arrays are described in WO 00/04382, WO 00/04389 and WO 00/04390; the disclosures of which are herein incorporated by reference. One application of particular interest in which the subject invention finds use is proteomics, in which the subject methods are used to characterize the proteome or some fraction of the proteome of a physiological sample, e.g. a cell, population of cells, population of proteins secreted by a cell or population of cells, etc. By proteome is meant the total collection or population of intracellular proteins of a cell or population of cells and the proteins secreted by the cell or population of cells. In using the subject methods in proteomics applications, the subject methods are employed to measure the presence, and usually quantity, of the proteins which have been expressed in the cell of interest, i.e. are present in the assayed physiological sample derived from the cell of interest. In certain applications, the subject methods are employed to characterize and then compare the proteomes of two or more distinct cell types.

The subject methods provide for a number of significant advantages over other array based hybridization assays in the above described and other applications. Specifically, the subject methods are based on the use of a universal array of tag complements, i.e. an array that is not specifically tailored to detection of specific analytes in a sample. Instead, specificity with regard to the types of analytes that are assayed by the arrays is provided by attaching identifying tags to the desired affinity ligands that correspond to the analytes of interest and using the tagged affinity ligands to assay the sample. As such, one can use the same universal array and corresponding set of tags in any analyte assay, with the specificity of analytes assayed being provided by the particular tagged affinity ligands that are employed. Furthermore, the subject methods overcome problems typically found in affinity ligand arrays, e.g. protein arrays, in which the affinity ligand is bound directly to the substrate surface when contacted with the sample, where such problems include: storage stability, problems in binding activity or efficiency and the like. More specifically, the subject methods provide for universal conditions for immobilization of the affinity ligand to a solid surface. In addition, the subject methods provide enhanced stability of the affinity ligands by performing the immobilization in liquid/solid phase, rather than by utilizing printing procedures which rely on covalent bond formation during drying of the affinity ligand solution on the solid surface. Furthermore, the subject methods provide a means of directed immobilization of the affinity ligands which are to be utilized for biological recognition—i.e. improved ratio between reactive affinity ligands vs. inactivated affinity ligands due to involvement of the binding sites of the affinity ligands in the immobilization process. Furthermore, the subject invention provides the means to perform real homogenous assays between the affinity ligands and the analytes followed by efficient, selective and quantitative entrapment of the ligand/analyte complexes on the array surfaces.

Kits

Also provided are kits for performing hybridization assays according to the subject invention. Such kits according to the subject invention include at least one of: (a) a tag complement or universal array; and (b) a set of tagged affinity ligands, where the tag portion of each member of the set of tagged affinity ligands corresponds to, i.e. is complementary to or has a sequence identical to a sequence found in, a tag complement on the array. In many embodiments, the kits include both the universal array and a set of tagged gene specific primers.

In addition to including at least one of the array and the set of tagged gene specific primers, the kits also include a means for determining the analyte, e.g. protein, to which each tag and tag complement on the array corresponds. In other words, the kits include a means for readily matching any given tag and tag complement pair with a specific protein or other analyte. Put another way, the kits include a means for readily identifying the location on the array that a specific tagged affinity ligand, and therefore tagged affinity ligand/analyte binding complex prepared therefrom, will hybridize during a hybridization assay. With this means, one can readily identify the location on the array that corresponds to a particular protein or other analyte of interest in the assay that is to be performed

This means for identifying the analyte to which a given tag-tag complement pair correspond may take a variety of forms, one or more of which may be present in the kit. One form in which this means may be present is as printed information on a suitable medium or substrate, e.g. a piece or pieces of paper on which the information is printed. Yet another means would be a computer readable medium, e.g. diskette, CD, etc., on which the information has been recorded. Yet another means that may be present is a website address which may be used via the internet to access the information at a removed site. Any convenient means may be present in the kits.

The kits may further comprise one or more additional reagents employed in the various methods, such as labeling reagents, various buffer mediums, e.g. hybridization and washing buffers, and the like.

It is evident from the above discussion that the subject methods provide for a significant advance in the field of ligand arrays, particularly protein arrays. The subject invention provides for the use of a single “universal array” in a plurality of different analyte detection assays which differ from each other with respect to the identity of the analytes being assayed. The same universal array can be manufactured and used in many different types of hybridization assays, thereby providing for ease in quality control, high throughput manufacture, and economical manufacture. In addition, problems with array stability, binding of affinity ligand to target analyte, differences is binding efficiencies between surface bound ligand and solution phase target analyte, etc, are avoided in the subject methods. Accordingly, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of detecting the presence of at least one analyte in a sample, said method comprising:
(a) producing at least one surface bound hybridization complex on the surface of an array of distinct tag complements immobilized on a surface of a solid support, wherein said surface bound hybridization complex comprises a tag complement hybridized to a tag, wherein said tag is part of a tagged affinity ligand that is bound to said analyte;
(b) detecting the presence said at least one surface bound hybridization complex; and
(c) relating the presence of said at least one surface bound hybridization complex to the presence of said at least one analyte in said sample to determine the presence of at least one analyte in a sample.

2. The method according to claim 1, wherein said producing step comprises:
(i) contacting said sample with a population of tagged affinity ligands under conditions sufficient to produce said at least one analyte/tagged affinity ligand complex; and
(ii) contacting said at least one analyte/tagged affinity ligand complex produced in step (i) with said array of tag complements under hybridization conditions to produce said at least one surface bound hybridization complex.

3. The method according to claim 1, wherein said tag and tag complements are nucleic acids.

4. The method according to claim 3, wherein the magnitude of any difference in hybridization efficiency between any two tag-tag complement pairs employed in said assay does not exceed about 10 fold.

5. The method according to claim 4, wherein the magnitude of any difference in hybridization efficiency between any two tag-tag complement pairs employed in said method does not exceed about 5 fold.

6. The method according to claim 5, wherein the magnitude of any difference in hybridization efficiency between any two tag-tag complement pairs employed in said method does not exceed about 3 fold.

7. The method according to claim 3, wherein any tag employed in said assay has a level of cross-hybridization that does not exceed about 10%.

8. The method according to claim 7, wherein any tag employed in said method has a level of cross-hybridization that does not exceed about 2%.

9. The method according to claim 8, wherein any tag employed in said method has a level of cross-hybridization that does not exceed about 1%.

10. The method according to claim 1, wherein said analyte is a polypeptide.

11. The method according to claim 10, wherein said polypeptide is a protein.

12. The method according to claim 1, wherein said tagged affinity ligands comprise an antibody or binding fragment thereof.

13. The method according to claim 1, wherein said tagged affinity ligands are labeled.

14. The method according to claim 1, wherein said method is a method of determining the presence of a plurality of analytes in said sample.

15. The method according to claim 14, wherein said plurality of analytes are proteins.

16. A kit for use in an analyte detection assay, said kit comprising:
(a) at least one of:
(i) an array of distinct tag complements immobilized on the surface of a solid support; and
(ii) a set of distinct tagged affinity ligands; and
(b) means for identifying the physical location on said array to which each distinct tagged affinity ligand of said set hybridizes.

17. The kit according to claim 16, wherein said kit comprises both said array and said set of tagged affinity ligands.

18. The kit according to claim 16, wherein the magnitude of any difference in hybridization efficiency between any two tag-tag complement pairs taken from said array and set of tagged affinity ligands does not exceed about 10 fold.

19. The kit according to claim 16, wherein any tag found in said set of tagged affinity ligands has a level of cross-hybridization with respect to said array that does not exceed about 10%.

20. The kit according to claim 16, wherein said means comprises a medium that includes: (a) identifying information about the physical location on said array to which each distinct tagged affinity ligand hybridizes; or (b) a means for remotely accessing said information.

21. The kit according to claim 20, wherein said means for remotely accessing said information is a website address.

22. An array of distinct tag complements immobilized on a solid support, wherein said tag complements are members of a collection of tag-tag complement pairs in which the magnitude of any difference in hybridization efficiency between any two tag-tag complement pairs in said collection does not exceed about 10 fold.

23. The array according to claim 22, wherein said tag complements are nucleic acids.

24. The array according to claim 22, wherein said array has a density that does not exceed about 400 spots/cm².

25. A set of distinct tagged gene affinity ligands comprising a tag domain and an affinity ligand, wherein said tag domains are members of a collection of tag-tag complement pairs in which the magnitude of any difference in hybridization efficiency between any two tag-tag complement pairs in said collection does not exceed about 10 fold.

26. The set according to claim 25, wherein any tag domain has a level of cross-hybridization with respect to said tag complements of said collection that does not exceed about 10%.

27. The set according to claim 25, wherein said set comprises at least 20 distinct tagged affinity ligands.
